(19) United States
(12) Patent Application Publication
(10) Pub. No.: US 2023/0008026 A1
(43) Pub. Date: Jan. 12, 2023

(54) ACOUSTOFLUIDIC CENTRIFUGE FOR NANOPARTICLE ENRICHMENT AND SEPARATION

(71) Applicant: Duke University, Durham, NC (US)

(72) Inventors: Yuyang Gu, Durham, NC (US); Tony Jun Huang, Durham, NC (US)

(21) Appl. No.: 17/719,307

(22) Filed: Apr. 12, 2022

Related U.S. Application Data
(60) Provisional application No. 63/173,738, filed on Apr. 12, 2021.

(51) Int. Cl. B01L 3/00; G01N 1/40; C12Q 1/6806; G01N 21/64; B01D 21/28

(52) U.S. Cl. CPC B01L 3/502761; G01N 1/4077; C12Q 1/6806; G01N 21/6428; B01D 21/283; G01N 2001/4094; B01L 2400/0436; B01L 2200/0652; G01N 2021/6439

(57) ABSTRACT

The present disclosure provides for acoustofluidic centrifuge systems that can enrich and separate nanoparticles disposed in a fluid, such as liquid droplets, in a fast and efficient manner. Exemplary systems include a sound wave generator, such as a pair of slanted interdigitated transducers, and a containment boundary, such as a PDMS ring. The sound wave generator can produce surface acoustic waves that are capable of driving droplets to spin in a manner that can separate different sized particles into groups. In some embodiments, the acoustofluidic centrifuge system can include a plurality of containment boundaries in fluid communication with each other, allowing particles to separate between the containment boundaries. Methods of operating such systems, including methods of isolating different exosome subpopulations, are also disclosed.

### CROSS REFERENCE TO RELATED APPLICATION

The present disclosure claims priority to and the benefit of U.S. Provisional Patent Application No. 63/173,738, entitled “Acoustofluidic Centrifuge for nanoparticle Enrichment and Separation,” filed on Apr. 12, 2021, the disclosure of which is incorporated by reference herein in its entirety.

### FEDERAL FUNDING LEGEND

This invention was made with Government support under Federal Grant Nos. UG3TR002978, R01GM132603, R01GM135486, R33CA223908, R01GM127714, and R01HD086325 awarded by the National Institutes of Health; Federal Grant No. W81XWH-18-1-0242 awarded by the Army Medical Research Acquisition Activity; and Federal Grant No. ECCS-1807601 awarded by the National Science Foundation. The Federal Government has certain rights to this invention.

### FIELD

The present disclosure relates using acoustofluidics to manipulate nanoparticles, and more particularly relates to an acoustofluidic centrifuge able to rotate nano-sized particles in a manner that allows the particles to be enriched and separated in an effective manner.

### BACKGROUND

Nanoparticle manipulation is of great importance in a variety of biomedical and biochemical applications, including gene/drug delivery, precision bioassays, cancer diagnosis, and catalyzing reactions. As such, the ability to perform nanoparticle concentration or separation, or achieve self-assembly of nanostructures, has emerged as a prominent interdisciplinary need in many fields. Additionally, although there is a strong desire for controlling nanoscale (approximately less than 100 nanometers) objects, only a handful of methods can achieve manipulation at this level. Conventional techniques for nanoscale manipulation include ultracentrifugation, nanopore filtration, dielectrophoresis, magnetopheresis, optical tweezing, and plasmonic tweezing. While each of these methods has certain advantages depending on the given application, there are still many drawbacks associated with their routine use. For example, ultracentrifugation and filtration-based manipulation have relatively low sample yields and require long processing periods, and while optical and plasmonic tweezers provide high precision, these approaches are usually restricted to manipulating a relatively small number of particles, thus severely limiting their practical applications.

To the extent other methods to capture and control nanoparticle materials have been tried, such as using acoustic-based systems, such systems have been limited in their ability to fully control the nano-sized particles. The produced acoustic radiation force of existing systems is woefully insufficient to achieve meaningful separation and control of the nanoparticle materials. The acoustic waves as produced using systems as currently designed are unable to control microparticles on the order of tens of nanometers. Rather, they are more suited for manipulation on a sub-micron scale (greater than 100 nanometers).

Accordingly, there is an ongoing opportunity or need for improved methods for capturing and controlling nanoparticle materials.

### SUMMARY

The Summary is provided to introduce a selection of concepts that are further described below in the Detailed Description. This Summary is not intended to identify key or essential features of the claimed subject matter, nor is it intended to be used as an aid in limiting the scope of the claimed subject matter.

The present disclosure provides for an acoustofluidic centrifugation technique that leverages an entanglement of acoustic wave actuation and the spin of a fluidic droplet to enable nanoparticle enrichment and separation. By combining acoustic streaming and droplet spinning, rapid (less than approximately one minute) nanoparticle concentration and size-based separation can be achieved. The resulting resolution can be sufficient to identify and isolate exosome subpopulations, among other uses provided for herein. The underlying physical mechanisms have been characterized both numerically and experimentally, and the ability to process biological samples (e.g., DNA segments, exosome subpopulations, etc.) has been successfully demonstrated. Altogether, this acoustofluidic centrifuge disclosure overcomes existing limitations in the manipulation of nanoscale (less than approximately 100 nanometers) bioparticles, and can be valuable for a variety of applications in the fields of biology, chemistry, engineering, material science, and/or medicine.

The provided-for acoustofluidic centrifuge system can leverage acoustically driven spinning droplets to manipulate particles with sizes down to a few nanometers. Various functionalities of these systems and methods can include nanoparticle concentration, separation, and transport. The basic system can include a sound wave generator, such as a pair of slanted interdigitated transducers (IDTs), and a containment boundary, such as a circular polydimethylsiloxane (PDMS) containment ring, to encapsulate at least a portion of a droplet disposed within. The containment boundary can define a shape the at least a portion of a droplet disposed within the boundary.

Systems, and the related methods, of this nature can produce surface acoustic waves (SAWs) capable of driving droplets, or portion(s) thereof, disposed in the containment boundary to spin along a central axis of the droplet, or portion(s) thereof. More particularly, the SAWs can propagate in different directions, causing the droplet to spin. This spinning motion can initiate Stokes drift along a closed path, such as a circular closed path when the containment boundary is a circular ring, that can transfer momentum to the fluid in a manner that can significantly increase the inner streaming velocity and shear rate within the droplet, for example approximately in the range of about 10 times to about 100 times. Particles within this “rotational vortex field” can follow a helical trajectory and can be rapidly concentrated to a center of the droplet as a result of the combination of the acoustic radiation force and drag force. The systems and methods incorporating SAWS, thus, combine acoustic force (e.g., droplet spinning) and acoustic streaming to manipulate the droplets, or portion(s) thereof. In some instances, multiple droplets can be utilized simultaneously, such as by way of two containment boundaries, which can provide even further improved results.

Acoustic waves provided for in conjunction with the present systems and methods can rotate a liquid droplet, or portion(s) thereof, with a variable sample volume (from nanoliters to microliters) to influence nanoparticles of various sizes (from a few nanometers to a few micrometers). As a result, by way of non-limiting example, leveraging droplet resonance can enables 28 nm nanoparticles, as well as strands of DNA, to be concentrated within one minute or less. Additionally, in embodiments that include an acoustofluidic centrifuge system with dual rotating droplets, nanoparticles of various sizes, including exosome subpopulations, can be separated with high purity. A person skilled in the art will appreciate that purity in the present context is a population of one size of nanoparticles among the population of all the nanoparticles in a sample, and that separating with high purity, in at least some instances, can include separation of approximately 80% purity or greater. Use of these systems and methods in conjunction with isolating biological samples, such as different exosome subpopulations, appears to be a particularly useful development not previously achieved. Further, the comprehensive theoretical modelling and matching experimental results performed using the acoustic-mediated nanoparticle manipulation platforms and methods disclosed herein can be extrapolated to a variety of other applications, including but not limited to simplifying transfection, automating vesicle cargo loading, and/or accelerating liquid biopsies.

One exemplary embodiment an acoustofluidic centrifuge system includes at least one sound wave generator and at least one containment boundary. The containment boundary is configured to encapsulate at least a portion of a fluid droplet. The sound wave generator is configured to generate acoustic waves that propagate towards the at least one containment boundary to cause the at least a portion of a fluid droplet encapsulated in the boundary to spin along a central axis. The central axis can be that of the at least a portion of a fluid droplet and/or a central axis of the at least one containment boundary.

The at least one sound wave generator can include at least one interdigitated transducer. Alternatively, or additionally, the at least one sound wave generator can include at least one acoustic transducer. One or more of such interdigitated and/or acoustic transducers can be slanted in some instances. In some embodiments, the sound wave generator(s) can include a pair of opposed interdigitated and/or acoustic transducers. In such embodiments, each transducer of the pair can be disposed on opposite sides of the at least one containment boundary. One or more of such transducers can be slanted in some instances of this configuration as well.

The at least one containment boundary can include a circular ring. The at least one containment boundary can include one or more polymers, such as polydimethylsiloxane (PDMS). In some embodiments the at least one containment boundary can include two containment boundaries. The two containment boundaries can be in communication with each other by way of a channel disposed between the two boundaries. The two containment boundaries can both include a circular ring.

One exemplary method of separating nanoparticles includes generating at least one sound wave such that the sound wave propagates to at least one containment boundary having at least a portion of one fluid droplet disposed in the at least one containment boundary. The at least a portion of one fluid droplet has a plurality of nanoparticles disposed in it. The at least one sound wave causes the at least a portion of one fluid droplet to rotate. Further, rotation of the at least a portion of one fluid droplet causes at least one nanoparticle of the plurality of nanoparticles to travel along a first trajectory of a plurality of trajectories and at least a second nanoparticle of the plurality of nanoparticles to travel along a second trajectory of the plurality of trajectories, the first and second trajectories being different trajectories.

The action of generating at least one sound wave can further include providing current to at least one acoustic transducer, which can propagate at least one acoustic wave to the at least one containment boundary. Alternatively, or additionally, the action of generating at least one sound wave can further include providing current to at least one interdigitated transducer, which can propagate at least one acoustic wave to the at least one containment boundary. The at least one sound wave can deform a liquid-air interface of the at least a portion of one fluid droplet, which can result in the at least a portion of one fluid droplet rotating.

The at least one trajectory of the plurality of trajectories along which at least one nanoparticle of the plurality of nanoparticles travels can include a helical path. At least one of the at least one nanoparticle that travels along the first trajectory and the at least one nanoparticle that travels along the second trajectory travels along a vortex-shaped streamline. Further, the at least one nanoparticle(s) can be influenced by an angular momentum of the rotation of the at least a portion of one fluid droplet. In at least some embodiments, the rotation of the at least a portion of one fluid droplet can have a dual-axis rotational trajectory.

The method can include controlling an acoustic streaming speed of the at least a portion of one fluid droplet. The can be achieved, for example, by controlling at least one of an acoustic wave amplitude, a frequency of an acoustic wave, an angle at which an acoustic wave is delivered, and a location of the acoustic wave. Controlling an acoustic streaming speed can further include adjusting a size of the at least one containment boundary and/or a volume of the at least one containment boundary. Travel along the first trajectory or the second trajectory (as well as other trajectories) can be based on a size of the nanoparticles of the plurality of nanoparticles.

In some embodiments the at least one containment boundary can include a plurality of containment boundaries in fluid communication with each other. The plurality of containment boundaries can include a first containment boundary and a second containment boundary. The action of at least one sound wave causing the at least a portion of one fluid droplet to rotate can further include causing a first at least a portion of one fluid droplet to rotate in the first containment boundary and causing a second at least a portion of one fluid droplet to rotate in the second containment boundary. Further, the action of causing at least a first nanoparticle of the plurality of nanoparticles to travel along a first trajectory and causing at least a second nanoparticle of the plurality of nanoparticles to travel along a second trajectory can include passing at least one of the first and/or second nanoparticles through a channel disposed between the first and second containment boundaries. In at least some embodiments, the first nanoparticle(s) can have a different size than the second nanoparticle(s) such that a different sized nanoparticle(s) travels along a different trajectory of the first and second trajectories.

An exemplary method of isolating different extracellular vesicle subpopulations includes mixing DNA strands disposed in at least a portion of a fluid droplet with a fluorescent marker, intercalating the at least a portion of a fluid droplet such that the DNA strands are able to express a fluorescent signal, and activating an acoustic signal to activate the fluorescent marker in a manner such that concentrated DNA strands express an amplified fluorescent signal.

In at least some embodiments, activating an acoustic signal to activate the fluorescent marker in a manner such that concentrated DNA strands can express an amplified fluorescent signal can include operating at least one sound wave generator to produce the acoustic signal. In some such embodiments, the at least a portion of a fluid droplet can be encapsulated by at least one containment boundary. Further, the acoustic signal can be effective to cause the at least a portion of a fluid droplet to spin along a central axis. The central axis can be of the at least a portion of a fluid droplet and/or the containment boundary.

Activating an acoustic signal can include causing the at least a portion of a fluid droplet to spin. Additionally, or alternatively, activating an acoustic signal can include generating at least one sound wave such that the sound wave propagates to at least one containment boundary having the at least a portion of one fluid droplet disposed in the containment boundary. In some embodiments, the fluorescent marker can include a SYTOX dye.

### BRIEF DESCRIPTION OF THE DRAWINGS

The accompanying Figures are provided by way of illustration and not by way of limitation. The foregoing aspects and other features of the disclosure are explained in the following description, taken in connection with the accompanying example figures (also “FIG.”) relating to one or more embodiments:

FIG. 1A is a perspective, schematic view of one exemplary embodiment of an acoustofluidic centrifuge;

FIG. 1B is a perspective detailed view of a portion of the acoustofluidic centrifuge of FIG. 1A;

FIG. 2A is a schematic top view of a droplet illustrating deformation during a spin motion, like the motion created by the acoustofluidic centrifuge of FIG. 1A;

FIG. 2B is a sequence of images illustrating a side view of a rotating droplet taken at selected periods of time in response to sound waves being propagated by an acoustofluidic centrifuge like the acoustofluidic centrifuge of FIG. 1A;

FIG. 3A is a schematic illustration of how acoustic parameters can impact particle trajectory;

FIG. 3B is a schematic illustration of a numerically simulated particle trajectory within a spinning droplet;

FIG. 3C are fluorescent images both before (left) and after (right) an acoustic field is turned on for the spinning droplet of FIG. 3B;

FIG. 4 is a schematic flowchart illustrating one exemplary embodiment of a process of DNA enrichment and fluorescent signal enhancement in a spinning droplet;

FIG. 5 is a schematic illustration of numerical simulation results showing a difference in nanoparticle trajectories for two particles having different sizes;

FIG. 6A is a top, schematic view of one exemplary embodiment of a portion of a dual-droplet acoustofluidic centrifuge;

FIG. 6B is a composite image showing a particle trajectory through a center channel of the dual-droplet acoustofluidic centrifuge of FIG. 6A;

FIG. 7A is a fluorescent image before an acoustic signal is turned on for the dual-droplet acoustofluidic centrifuge of FIG. 6A;

FIG. 7B is a fluorescent image after an acoustic signal is turned on for the dual-droplet acoustofluidic centrifuge of FIG. 6A;

FIG. 8 is a graph illustrating a particle size distribution comparison between pre-separation samples and post-separation samples for the dual-droplet acoustofluidic centrifuge of FIG. 6A;

FIG. 9A is a perspective view of a simulated 3D flow field of another embodiment of a dual-droplet acoustofluidic centrifuge;

FIG. 9B illustrate flow fields at different z-planes in a microchannel of the dual-droplet acoustofluidic centrifuge of FIG. 9A;

FIG. 10A is a side, schematic view of the dual-droplet acoustofluidic centrifuge of FIG. 6A with one droplet (right) with a sample and one blank droplet (left);

FIG. 10B is the side, schematic view of the dual-droplet acoustofluidic centrifuge of FIG. 10A with acoustic waves exciting particles of the droplet with the sample;

FIG. 10C is the side, schematic view of the dual-droplet acoustofluidic centrifuge of FIG. 10A after a separation and transport process has occurred such that particles with different sizes are immersed in different droplets;

FIG. 11A is a side, schematic view of a three-droplet acoustofluidic centrifuge with one droplet (right) with a sample, one blank droplet (middle), and a second blank droplet (left);

FIG. 11B is the side, schematic view of the three-droplet acoustofluidic centrifuge of FIG. 11B with acoustic waves exciting particles of the droplet with the sample; and

FIG. 11C is the side, schematic view of the three-droplet acoustofluidic centrifuge of FIG. 11B with acoustic waves further exciting particles of one of the previously blank droplets (middle) that now includes particles therein.

### DETAILED DESCRIPTION

Certain exemplary embodiments will now be described to provide an overall understanding of the principles of the structure, function, manufacture, and use of the devices and methods disclosed herein. One or more examples of these embodiments are illustrated in the accompanying drawings. Those skilled in the art will understand that the devices and methods specifically described herein and illustrated in the accompanying drawings are non-limiting exemplary embodiments and that the scope of the present disclosure is defined solely by the claims. The features illustrated or described in connection with one exemplary embodiment may be combined with the features of other embodiments. Such modifications and variations are intended to be included within the scope of the present disclosure.

Articles “a” and “an” are used herein to refer to one or to more than one (i.e., at least one) of the grammatical object of the article. By way of example, “an element” means at least one element and can include more than one element. “About” is used to provide flexibility to a numerical range endpoint by providing that a given value may be “slightly above” or “slightly below” the endpoint without affecting the desired result. The use herein of the terms “including,” “comprising,” or “having,” and variations thereof, is meant to encompass the elements listed thereafter and equivalents thereof as well as additional elements. As used herein, “and/or” refers to and encompasses any and all possible combinations of one or more of the associated listed items, as well as the lack of combinations where interpreted in the alternative (“or”).

Moreover, the present disclosure also contemplates that in some embodiments, any feature or combination of features set forth herein can be excluded or omitted. To illustrate, if the specification states that a complex comprises components A, B and C, it is specifically intended that any of A, B, or C, or a combination thereof, can be omitted and disclaimed singularly or in any combination. Similarly, to the extent features or steps are described herein as being a “first feature” or “first step,” or a “second feature” or “second step,” such numerical ordering is generally arbitrary, and thus such numbering can be interchangeable. Moreover, a person skilled in the art will appreciate that not all of the method steps disclosed herein are required, and, in view of the present disclosure, will understand how modifications can be made to each step, the order of the steps, the limitation of certain steps, etc. without departing from the spirit of the present disclosure while still achieving the desired goals.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as approximately in the range of about 1% to about 50%, it is intended that values such as approximately in the range of about 2% to about 40%, approximately in the range of about 10% to about 30%, or approximately in the range of about 1% to about 3%, etc., are expressly enumerated in this specification. These are only examples of what is specifically intended, and all possible combinations of numerical values between and including the lowest value and the highest value enumerated are to be considered to be expressly stated in this disclosure, as are values slightly above and/or slightly below those ranges at least in instances in which the term “about” is used. A number of terms may be used throughout the disclosure interchangeably but will be understood by a person skilled in the art.

Unless otherwise defined, all technical terms used herein have the same meaning as commonly understood by one of ordinary skill in the art to which this disclosure belongs. Additionally, like-numbered components across embodiments generally have similar features unless otherwise stated or a person skilled in the art would appreciate differences based on the present disclosure and his/her knowledge. Accordingly, aspects and features of every embodiment may not be described with respect to each embodiment, but those aspects and features are applicable to the various embodiments unless statements or understandings are to the contrary.

One aspect of the present disclosure provides an acoustofluidic centrifuge system, and related methods for using the same, that uses acoustically driven spinning droplets to manipulate particles with sizes down to a few nanometers. The system can comprise a sound wave generator, such as at least one transducer (often times a pair of transducers), and one or more containment boundaries, such as one or more rings. More particularly, in some embodiments the transducer(s) can include slanted interdigitated transducers (IDTs) and the containment boundary(ies) can include a containment ring, such as a polydimethylsiloxane (PDMS) containment ring. The containment ring can be a circular ring configured to retain a fluid droplet in an approximately hemispherical dome shape.

The droplet can provide a medium for an analyte (e.g., micro- to nano-sized particles). The fluid, or fluid-like, droplet(s) can be any suitable liquid or other material capable of creating the desired particle movement. The volume of the droplet can be varied according to the application, ranging from nanoliters to microliters.

The IDTs can be configured to generate surface acoustic waves (SAWs), which can propagate toward the droplet and drive the droplet to spin along a central axis. This spinning motion can initiate transfer momentum to the fluid in a manner that increases the inner streaming velocity and shear rate within the droplet. Particles within the droplet can follow a corresponding helical trajectory and can be rapidly concentrated to a center of the droplet as a result of the combination of the acoustic radiation force and drag force. The spin rate can be tuned to accommodate various analytes, for example, by changing a volume of the droplet and/or the size of the PDMS containment ring, sometimes referred to as a confinement ring.

In some embodiments, an acoustofluidic centrifuge system can include two or more PDMS rings that can be connected by a microchannel, and one or more pairs of IDTs. By using dual rotating droplets in close proximity to one another, it can be possible to separate nanoparticles of various sizes by moving particles from one droplet to another. A dual-droplet system can also be further expanded to a multistage nanoparticle separation or enrichment system in which multiple groups of nanoparticles can be separated into different outlets in one device.

The systems and method disclosed herein advantageously allow analytes such as nanoparticles, exosomes, strands of DNA, etc., to be concentrated within a short period of time (e.g., less than one minute). The system can be adapted for a variety of applications, such as simplifying transfection, automating vesicle cargo loading, and/or accelerating liquid biopsies.

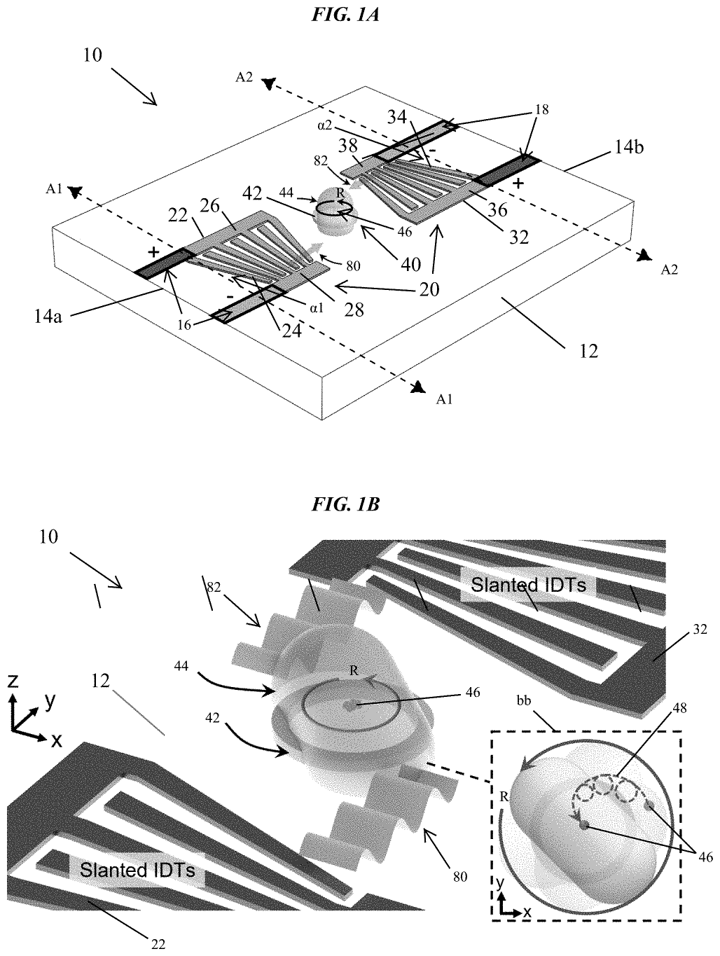

FIGS. 1A and 1B illustrate one embodiment of an acoustofluidic centrifuge system or device 10. The system 10 can be disposed on a substrate 12, and can include at least one sound wave generator 20, also referred to as an acoustic wave generator, and at least one containment boundary 40. In the illustrated embodiment, the sound wave generator 20 comprises a pair of opposed interdigitated transducers (IDTs) 22, 32, with the containment boundary 40, as shown a ring 42, disposed therebetween. The IDTs 22, 32 can be coupled to, formed on or in, or otherwise disposed with respect to the substrate 12 using a variety of techniques known to those skilled in the art for associating an IDT with a substrate. For example, in the illustrated embodiment, the IDTs 22, 32 are printed onto the substrate 12. As shown, the IDTs 22, 32 can be slanted, meaning one or more fingers 24, 34 extending from a first leg 26, 36 of the IDT 22, 32 and towards a second leg 28, 38 of the IDT 22, 32 can form an oblique angle α₁, α₂ with respect to the first leg 26, 36 when a straight axis A₁, A₂ substantially parallel with an edge 14a, 14b of the substrate 12 is defined for purposes of defining the oblique angle α₁, α₂. The fingers 24, 34 can extend at different angles with respect to each other and the straight axes A₁, A₂, and thus the oblique angle α₁, α₂ formed by one finger 24, 34 need not be the same as the oblique angle α₁, α₂ formed by other fingers 24, 34 of the same IDT 22, 32. The slanted IDTs 22, 32 enable broadband frequency excitation due to the varied finger spacing across the widths of the transducers 22, 32. More particularly, the slanting configuration of the IDTs 22, 32 allow waves 80, 82 to propagate at different positions than, for example, non-slanted IDTs.

The first and second legs 26, 28 and 36, 38 can extend substantially parallel to each other, though they do not have to be parallel with respect to each other, and can connect to a power supply, as represented by +/− lines 16, 18. Any suitable power supply can be used, including but not limited to linear power supplies, switched power supplies, and battery-based power supplies, and such power supplies can provide alternating current and/or direct current as desired. The power supply can be coupled to and/or provided as part of the substrate 12, and/or the power supply can be “off-chip,” meaning it can be disposed separate from the substrate 12 but is still in electrical communication with the sound wave generator 20. The power supply can provide current across the IDTs 22, 32, in turn propagating sound waves 80, 82, also referred to as acoustic waves, along the substrate 12 across each respective IDT 22, 32 and towards the opposed IDT 32, 22. A voltage provided by the power supply can be approximately in the range of about 1 Volt to about 300 Volts, although those amounts are by no means limiting. The amount of voltage supplied, and the amount of current that is provided, can vary based on a variety of factors, including but not limited to the IDT design. As shown, because the containment boundary 40 is disposed between the IDTs 22, 32, the resulting sound waves 80, 82 propagate towards the containment boundary 40, and a droplet 44 disposed therein. As shown more particularly in FIG. 1B, the sound waves 80, 82 can be surface acoustic waves (SAWs). While in the illustrated embodiment two IDTs 22, 32 are provided, in alternative embodiments only one IDT may be provided, or more than two IDTs may be provided. However, a person skilled in the art, in view of the present disclosures, will appreciate that the use of one IDT, for example, may result in a center axis of a droplet portion not being at a center of the droplet portion. Further, other sound wave generators can be used in lieu or in addition to IDTs, such as piezoelectric actuators (including actuators having different actuator shapes and/or using various piezoelectric materials, such as an ultrasonic emitter, an air transducer, a bolt-clamped Langevin transducer, or a speaker), a mechanical vibrator, an optical laser, which can produce laser-induced acoustic waves, and an acoustic phased array. IDTs can sometimes more generally be referred to as acoustic transducers. A person skilled in the art will appreciate other types of acoustic transducers and/or other types of interdigitated transducers beyond IDTs.

The containment boundary 40, as shown a containment ring 42, can be disposed between the IDTs 22, 32. In the illustrated embodiment, it is disposed approximately centrally between the IDTs 22, 32 and approximately centrally with respect to the substrate 12, although other locations are possible. A central location, however, can assist in balancing forces in a design like the illustrate acoustofluidic system 10. The containment boundary 40 can be designed or otherwise configured to encapsulate a portion of a droplet 44, up to an entire droplet as shown, or even more than one droplet. It can also be designed to or otherwise configured to define a shape of the droplet portion 44. The containment boundary 40 confines the boundary of the fluid such that, when a droplet portion 44 (e.g., water droplet) is added to the containment boundary 40, it forms its equilibrium hemispherical shape when gravity and surface tension forces are balanced. It can be advantageous for the defined shape of the droplet 44 to be circular, or substantially circular, and thus often the containment boundary 40 can be circular, such as the illustrated ring-shape. The containment boundary 40 can be made of one or more materials. While there are many suitable materials, including but not limited to polymers and/or plastics, in one exemplary embodiment the containment boundary 40 can be made of PDMS.

The containment boundary 40 can be attached to the substrate 12 using a variety of techniques known to those skilled in the art, but in the illustrated embodiment, an adhesive is used to couple the containment boundary 40 to the substrate 12. The droplet portion 44 can be disposed within the containment boundary 40 using any known techniques for dispensing a droplet to a desired location, such as a pipette or syringe.

As illustrated in FIG. 1B, as the SAWs 80, 82 propagate towards the containment boundary 40 and into the droplet portion 44, entering two flanks of the droplet portion 44, the liquid-air interface can be deformed by the acoustic radiation pressure, thus causing the droplet portion 44 to rotate or spin, as shown in a direction R. In the illustrated embodiment the direction R of spinning is in a counterclockwise direction, although the spinning direction can be clockwise in other embodiments. As a result, one or more nanoparticles 46 disposed within the droplet portion 44 can begin to move. An inset bb of FIG. 1B illustrates a helical path or trajectory 48 that one such particle 46 can take as the droplet portion 44 rotates in the x-y plane. The path 48 can be induced due, at least in part, to the influence of both vortex streaming and the spinning droplet.

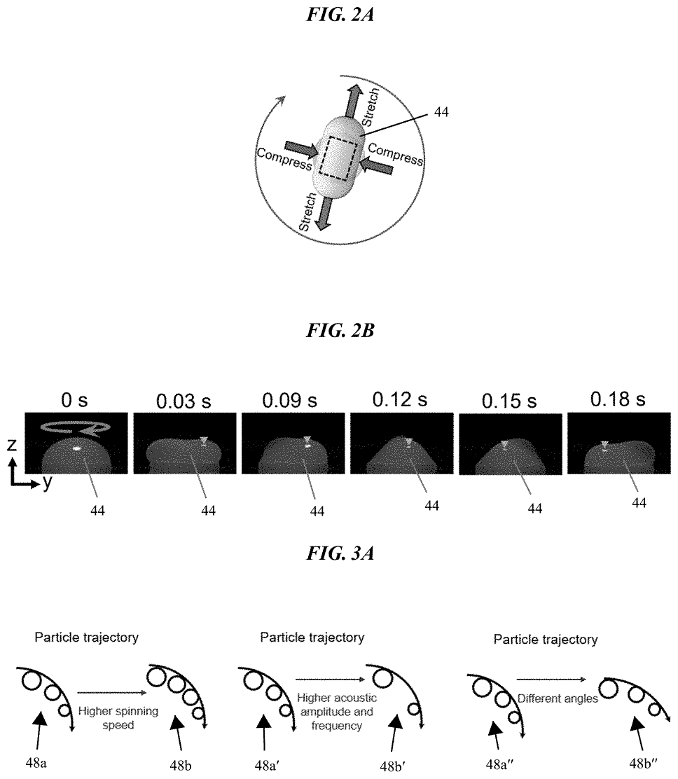

FIG. 2A provides a schematic illustration of droplet deformation during spin motion. As shown, compression and stretching can happen to the droplet portion 44 simultaneously during spinning. FIG. 2B provides a sequence of images showing the side view of a 30 μL rotating droplet portion 44. The SAW is activated at 0 s. The sequence shows that as the droplet portion 44 starts spinning, it stretches out to a concave ellipsoid shape, as illustrated at 0.03 seconds. The arrow in each image indicates a reference position that rotates along with the spinning droplet portion 44.

The droplet portion 44 spinning can experience three regimes from a non-spinning mode to a stable spinning mode. First, with a small acoustic amplitude excitation, internal vortex streaming can be generated while the droplet portion 44 remains in its equilibrium shape due to insufficient acoustic radiation pressure acting on the interface and the intensity of acoustic streaming. Second, as the acoustic amplitude increases, the length of the acoustic wave propagation before being fully attenuated can become longer and the vortex streaming can be enhanced as well. These effects can tend to break the equilibrium of the liquid-air interface while bottom and side boundaries of the droplet portion 44 can remain constrained within the containment boundary 40, which can result in slight oscillations of the fluid surface. Third, as momentum accumulates and the surface tension of the droplet portion 44 attempts to remain in balance with the acoustic radiation pressure and centrifugal force, the droplet portion 44 can gradually deform into a concave ellipsoid shape and can reach a stable spin mode with a periodic rotational boundary deformation, forming a “rotational capillary wave” that propagates along a free surface of the droplet portion 44. In this mode, particles 46 within the droplet portion 44 tend to migrate towards a center of the droplet portion 44, following a dual-axis rotational trajectory. One axis is with respect to the droplet 44 and the other axis is with respect to the particle. This dual-axis particle trajectory can follow a helical path 48 with the particle 46 itself also rotating, for example in the direction R, as shown in the inset bb of FIG. 1B. As shown, the dual-axis is both an approximate center of the droplet portion 44 and an approximate center of the helical path 48, each axis extending in and out of the page. Prior to the present disclosures, droplet portions were not spun in this manner, at least not in conjunction with attempting to separate and sort nanoparticles.

As noted above, while the present disclosure contemplates utilizing sound wave generators 20 like the IDTs 22, 32 to spin the droplet portion 44, a person skilled in the art will appreciate there are other ways by which spinning of the droplet portion 44 can be induced.

As indicated in FIG. 2B, compared to the original spherical cap shape, the spinning droplet 44 has a lower height but a higher maximum equatorial radius due, at least in part, to stretching in the horizontal direction. From a top view, the equilibrium cross-section of the droplet 44 can be a spinning, two-lobed, elliptical shape, with the droplet radial distance being at a fixed location over time. The rotational speed of the spinning droplet 44 can be calculated via a Fourier transform of a plotted waveform accounting for normalized distance with respect to time. The peaks of the waveform can correspond to twice the rotational frequency because the dual lobes of the single droplet 44 can cross a detection line two times each cycle. Extracting the droplet 44 speed from the waveform, the spin rate can be compared to classical droplet oscillation dynamics, as shown by Equation 1:

$${ω}_{n}^{2} = n ( n + 1 ) ( n - 1 ) ( n + 2 ) \frac{σ}{{r}^{3} [ ( n + 1 ) {ρ}_{liquid} + n {ρ}_{a i r} ]} \qquad ( 1 )$$

where n is the spherical harmonic degree that corresponds to the lobe number during spin, r is the radius of the droplet portion 44 (e.g., the radius of the spherical cap), a is the surface tension, and ρ_{liquid} and ρₐᵢᵣ are the density of the liquid and air, respectively. This oscillation equation can calculate the free oscillation frequency of a spherical droplet portion whose lobes repeatedly stretch and contract without spinning, as opposed to the continuous rotational deformation observed in the presently disclosed platform 10. The radius of the droplet portion 44 can be calculated from the volume of the droplet portion 44; a pipette can be used to generate a droplet portion with the known volume V, and given that the containment boundary 40 can be modeled as having a radius a and a height h, the radius of the droplet portion 44 can be calculated using the following relationship in Equation 2:

$$V - π {a}^{2} h = \frac{π}{3} {( r + \sqrt{{r}^{2} - {a}^{2}} )}^{2} [ 3 r - ( r + \sqrt{{r}^{2} - {a}^{2}} ) ] \qquad ( 2 )$$

For example, for a containment boundary that is a PDMS ring with a radius of 1.00 mm and a height of 0.55 mm, a 10.0 μL droplet has a radius of approximately 1.29 mm. There is a consistent correlation between the measured spin speed and the oscillation speed calculated using Equation 1. Thus, the spinning droplet portion 44 and the standard oscillatory motion can be similar, where the droplet portion 44 is forced from its equilibrium state in both cases. In some experiments, droplets with volumes approximately in the range of about 60 nL to about 90 μL were tested, which correspond to droplet radii approximately in the range of about 0.3 mm to about 3.6 mm.

Spin can generally be excited over a wide range of frequencies as long as the acoustic wave enters the droplet portion 44 from a position that has a slight bias from its center line. Further, frequency changes will not generally cause a major change to the spin speed. This is understandable at least because the rotational speed is primarily determined by the properties of the droplet portion 44 instead of the external excitation (as per Equation 1). On the other hand, as the applied power is increased, the droplet portion 44 will typically initially maintain its equilibrium shape, and then start to experience small oscillations until the acoustic power reaches a threshold value. At this threshold, the droplet 44 can enter its stable spinning mode, with the updated equilibrium shape (e.g., a concave ellipsoid shape) being further stretched with increased power, while the spin rate will not typically be affected. Notably, with higher droplet volumes, higher-order spin modes can be observed when the droplet 44 begins to spin, with four or six lobed droplets forming during rotation.

Turning to the motion of particles 46 within the spinning droplet 44, SAWs 80, 82 can induce acoustic streaming vortices inside the droplet portion 44. Further, particles 46 can be subject to both a drag force, which can arise from acoustic streaming, and an acoustic radiation force. To the extent these aspects have been studied prior to the present disclosure, it is believe to primarily relate to microparticles within a droplet that has a static shape. Within a spinning droplet, unlike the curvilinear particle path in a traditional SAW-driven droplet, particles (e.g., nanoparticles) move not only along the vortex-shaped streamline, but are also influenced by the angular momentum of the continuously rotating droplet. Experimentally, it was found that particles move along helical trajectories that correspond to a Stokes drift effect, while the localized spinning motion causes the water wave at the liquid-air interface to propagate along a circular path and results in a rotating Stokes drift. As a result, the particles 46 can follow a helical-shaped trajectory, such as the path 48 shown in the inset bb of FIG. 1B. Further experimental observations demonstrate that particles 46 generally travel along a larger vortex path, with multiple smaller circular movements along the path to the droplet center. Notably, the frequency of the smaller scale circular motion appears to be equal, or at least substantially equal, to the frequency of the bulk droplet spin, meaning the particle makes one local rotation while simultaneously moving closer to the global center of the droplet along its helical path during each rotation of the droplet.

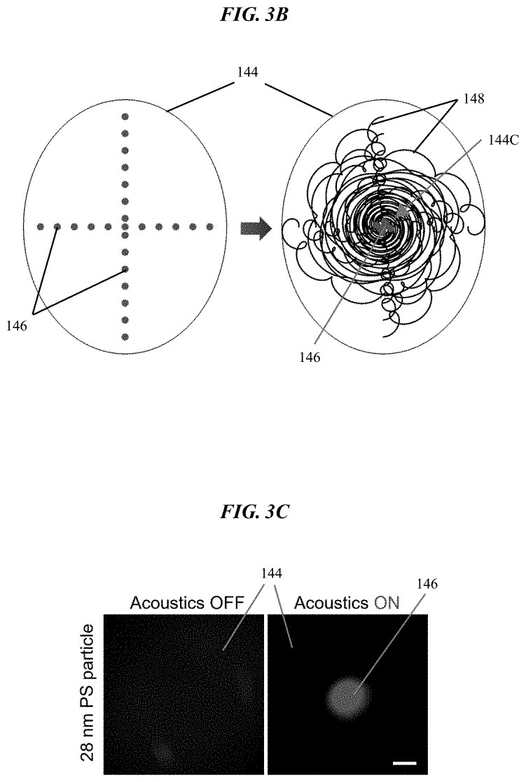

The trajectory or path at which a particle travels can be controlled by a variety of factors, including but not limited to droplet spinning speed and/or acoustic parameters. Acoustic parameters can include acoustic wave amplitude, frequency, an angle at which the wave is delivered, and/or a location of the wave. Streaming velocity and direction can be controlled by each of these parameters, which in turn can allow for particle trajectories or paths to be controlled. FIG. 3A illustrates three non-limiting examples of ways by which adjusting acoustic parameters can change a particle trajectory. In a first example, increasing spinning speed can yield a smaller “thread pitch” for a resulting helical particle trajectory 48b, that is there is an additional curl in the helical particle trajectory 48b as compared to a helical particle trajectory 48a from a lower spinning speed. By way of further example, increasing an acoustic amplitude and/or frequency can yield a larger “thread pitch” for a resulting helical trajectory 48b′ as compared to a helical particle trajectory 48a′. In a third, still non-limiting example, different angles of acoustic wave delivery can change a radius of the trajectory, as shown by a resulting helical particle trajectory 48b″ having a different angle as compared to a helical particle trajectory 48a″. The angle can be measured, for example, relative to an axis that is substantially aligned or parallel to the illustrated arrow between the two trajectories 48a″, 48b″, with the angle formed by the trajectories 48a″, 48b″ being respective best-fit lines that one of ordinary skill in the art can determine. The spinning speed of the droplet portion can be controlled, at least in part, by a size of the droplet and the containment boundary. Thus, by adjusting a size (e.g., radius) and/or volume of the containment boundary, a radius of the droplet portion can be adjusted.

FIG. 3B illustrates a numerically simulated particle trajectory for particles 146 within a spinning droplet 144, with a left side of the image illustrating the particles 146 prior to spinning the droplet 144 and a right side of the image illustrating the particles 146 after rotation or spinning of the droplet 144 has commenced. As the droplet 144 starts to spin, the particles 146 that were initially randomly distributed inside the droplet, as shown on a left side of the image, follow substantially helical trajectories 148 until concentrated at an approximate middle or center 144C of the droplet 144, as shown on a right side of the image. Fluorescent images provided in FIG. 3C include both a before image of the droplet 144, on the left, when the acoustic field is turned off, and an after image of the droplet 144, on the right, when the acoustic field is turned on, which shows the enrichment of 28 nm polystyrene (PS) particles 146. The illustrated scale bar is 50 μm.

After quantifying the particle velocity inside the droplet 144, it was determined that the droplet spin and the resulting localized rotation trajectory have a more significant effect than simply modifying the trajectory of the particles as they travel toward the center 144C of the droplet 144. Further experimentation determined that at low power levels, velocities of the particles 146 between the experiment and simulation had a very small difference. However, the velocity difference became larger, with as much as an approximately 80 mm/s variance once a higher power was applied. Moreover, the shear rate inside a spinning droplet can increase and can be positively correlated with the spin speed. Compared to a non-spinning droplet in which a shear rate is only generated by vortex acoustic streaming, the shear rate of a spinning droplet increases several times over as the rotational rate increases. Further, unlike the normal acoustic-streaming-induced shear rate distribution that usually decreases rapidly away from a boundary, the shear rates within the spinning droplet can remain high near the droplet center. This can potentially explain the rapid concentration of the microparticles provided for herein because the droplet spinning can enhance both vortex streaming velocity and fluid deformation within a droplet. While the entire fluid domain can be shown as spinning, the essence of this spinning motion can be the boundary periodic deformation along the radial axis. This continuous boundary deformation can generate a secondary-flow along the radial direction, which can push the particles 146 into the inner orbit of the vortex streaming and further propel the particles 146 towards the center 144C. Although the particle displacement has a small oscillation along the radial direction due to the continuous stretching and compressing of the droplet 144, the overall impact is to push the particles 146 inward. Thus, as this inward pushing effect accumulates, nanoparticles 146 can be concentrated to the droplet center 144C.

Differential Concentration of Nanoparticles Via Acoustofluidic Centrifuge

For particles with diameters smaller than approximately 1μm in a droplet, the drag force generated by both acoustic streaming (tangential direction) and spinning-enhanced secondary-flow (radial direction) can play a significant role in driving the particles to move along the fluid streamlines. The particle trajectory and end position in a sessile droplet is mainly related to two factors: (1) the ratio between the attenuation length of the acoustic wave (Lₛ=ρₛcₛλₛ/ρ_{f}c_{f}) and the droplet radius (r_{d}), where ρₛ, ρ_{f}, cₛ, c_{f}, λₛ are the density, acoustic velocity of the substrate and fluid, and the wavelength of the SAW in the substrate, respectively; (2) the ratio between the particle size and the acoustic wavelength in the fluid (κ=k_{f}aₚ), where k_{f} is the wave number in the fluid, and ap is the radius of the particle. When the wave attenuation length is larger than the droplet radius (Lₛ>r_{d}), and the particle size is small compared to the wavelength in the fluid (κ<1), particles inside the vortex streaming field can move and concentrate to the center. When dealing with nanoparticles, the aforementioned secondary-flow induced by spinning motion can have a drag force component in the radial direction and can dominate the concentration that can gradually bring the particles inward. Both by numerical and experimental efforts, it was shown that nanoparticles 146 can be rapidly (within approximately one minute or less) concentrated in the spinning droplet 144, with particle sizes down to about 28 nm in diameter, as shown in FIGS. 3B and 3C.

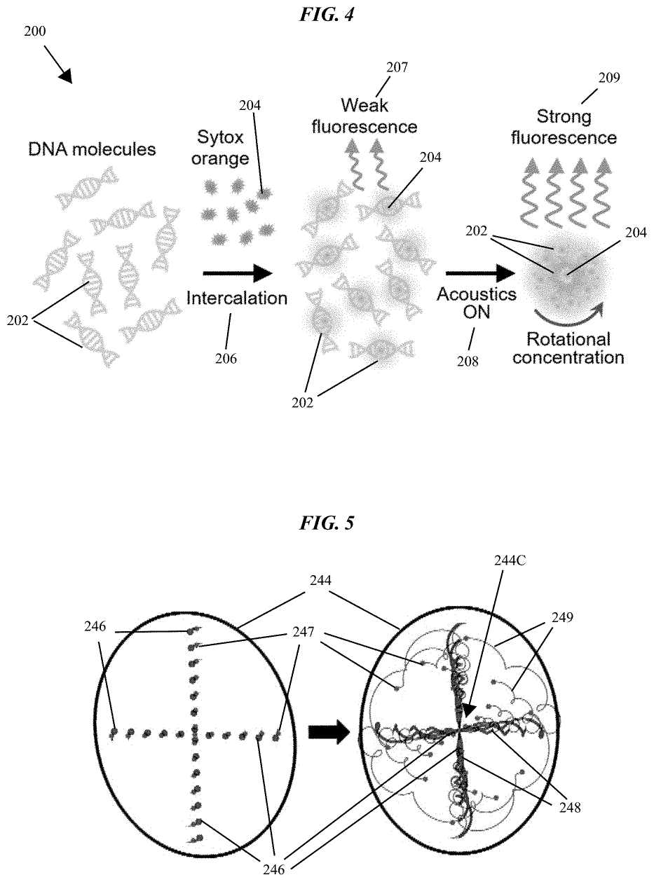

Rapid concentration of nanoparticles can enable various applications such as fluorescence based biospecimen detection. For instance, DNA molecules can be detected in acoustofluidic centrifuge systems provided for herein, or otherwise derivable from the present disclosures. This can be done, for example, with a fluorescent dye called SYTOX that enhances its fluorescence more than 500-fold upon intercalation with the DNA. A flow chart 200 provided for in FIG. 4 shows a process of DNA enrichment and fluorescent signal enhancement in a spinning droplet. The DNA strands and/or molecules 202 illustrated at a beginning of the flow chart 200 can be disposed in a droplet (not shown). More particularly, as shown in FIG. 4, by mixing the DNA molecules 202 in the droplet with a fluorescent marker 204, as shown SYTOX, this method can significantly enhance the fluorescent signal and detect the tagged DNA within a relatively short period (approximately 100 seconds). In the illustrated embodiment, SYTOX orange dye 204 (other dye colors can be used as well, such as green) is added into the sample droplet that contains the DNA molecules 202. After intercalation at action or step 206, the DNA molecules 202 can start to express a fluorescent signal. However, without concentration, the fluorescent signal can be too weak to be detected, as shown at state 207 in FIG. 4, representative of the DNA molecules 202 having weak fluorescence. In contrast, when an acoustic signal is generated, as shown at action or step 208, and droplet spin is initiated for the droplet 201, the DNA molecules 202 can be concentrated and the signal can be amplified, as shown at state 209 in FIG. 4, representative of the DNA molecules 202 within the droplet 201 having rotational concentration and the DNA molecules 202 having strong fluorescence. In order to assess the ability to avoid false positives, it was noted that while only using the SYTOX dye (without DNA molecules) in a spinning droplet, such as the droplet 201, there was no enhanced fluorescence observed. This proves the capability of the systems and methods provided for herein for DNA concentration as disclosed and described at least with respect to FIG. 4, or otherwise understood by a person skilled in the art in view of such disclosures and descriptions.

FIG. 5 illustrates numerical simulation results showing the difference in nanoparticle trajectories 248, 249 for particles 246, 247 with sizes of approximately 100 nm and approximately 28 nm. Similar to FIG. 3A, a left side of the image illustrates the particles 246, 247 prior to rotating or spinning a droplet 244 in which the particles 246, 247 are disposed, while a right side of the image illustrates the particles 246, 247 after rotation of the droplet 244 has commenced. As shown in the right side of the image, while the 100 nm particles 246 become concentrated in an approximate center 244C of the spinning droplet 244, following the trajectory or path 248, the 28 nm particles 249 follow the helical trajectory or path 249 but remain randomly distributed throughout the droplet 244. The shape of the trajectory or path 248 can also be helical, although, as shown, it is more direct, moving the particles 246 to the approximate center 244C quickly.

In addition to the rapid enrichment of nanoparticles, the system is capable of differentially concentrating nanoparticles of varying sizes. The interplay of the acoustic parameters (e.g., frequency and amplitude) and the droplet dimensions can generate different particle trajectories for different sized particles within the same droplet. As the combined radial force continuously pushes the particles towards the droplet center, different particles can share the same final equilibrium position (center region) while the time scale and migration speed for reaching this position can be different. Specifically, when nanoparticles with two different sizes are contained within a spinning droplet, the larger particles can experience higher acoustic radiation forces and drag forces, and smaller effects from Brownian motion. Numerical simulations were conducted to show that nanoparticles can be differentially concentrated with a small size difference (e.g., the separation of 28 nm diameter particles 247 and 100 nm diameter particles 246, as shown in FIG. 5). More specifically, FIG. 5 illustrates numerical simulation results showing the difference in nanoparticle trajectories 248, 249 for particles 246, 247 with sizes of 100 nm and 28 nm, respectively. While the 100 nm particles 246 become concentrated in the approximate center 244C of the spinning droplet 244, the 28 nm particles 247 follow the helical trajectory or path 249 but remain randomly distributed throughout the droplet 244. Experimental results verified this effect, as the 100 nm polystyrene particles were concentrated to the center of the droplet while the 28 nm polystyrene particles remained randomly dispersed throughout the droplet. Accordingly, a trajectory of the particles can be estimates based on one or more of an initial position of the particle(s), a spinning speed of the droplet portion (and thus the particle(s)), and/or an acoustic streaming speed.

Dual-Droplet Acoustofluidic Centrifuge

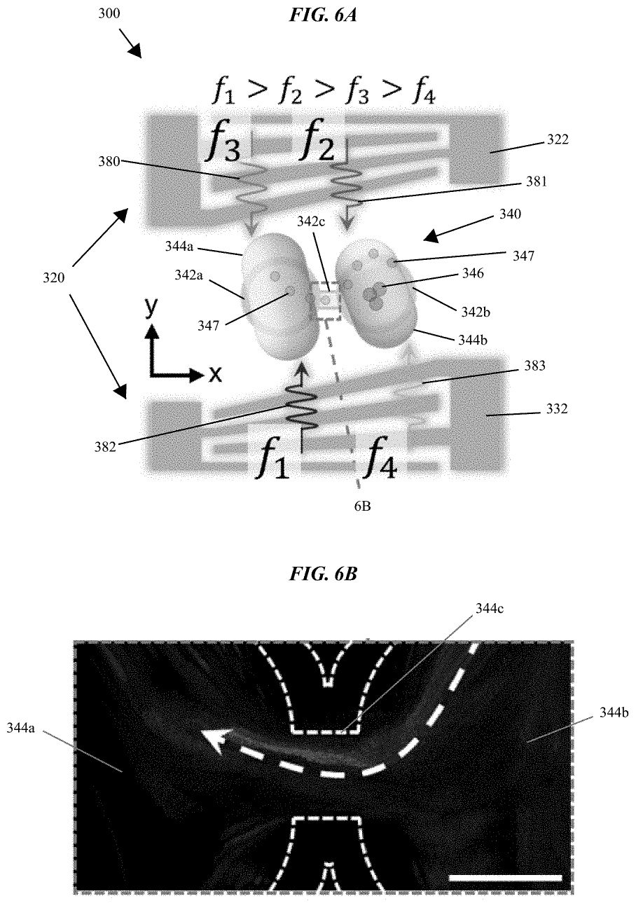

Although nanoparticles of different sizes can be differentially concentrated within a single droplet by concentrating the larger particles to the middle, this single-droplet acoustofluidic centrifuge device can impact the purity of the target nanoparticles with distinctive sizes because differential concentration and retrieval of the subsets of nanoparticles is conducted within the same droplet. To address this concern, multi-droplet based acoustofluidic centrifuge systems and devices, such as a dual-droplet based acoustofluidic centrifuge system or device, is provided that is practical, for example, for nanoparticle separation applications. FIG. 6A provides for an acoustofluidic centrifuge system or device 300 for particle separation and transport having a containment boundary 340 that includes two individual spinning droplet units 342a, 342b connected by a microchannel 342c, also referred to as a channel, to provide fluid communication therebetween, thus allowing for passage of particles 346, 347 disposed in the droplet(s) 344a, 344b. Similar to the system 10 of FIGS. 1A-1B, sound wave generators 320, as shown opposed slanted IDTs 322, 332, can be disposed on opposite sides of the containment boundary 340. In the illustrated embodiment, the containment boundary 340 comprises a first containment boundary, as shown a first ring 342a, and a second containment boundary, as shown a second ring 342b, in fluid communication with each other by way of the microchannel 342c. The droplets 344a, 344b respectively disposed in each ring 342a, 342b can become connected, or can be considered to be connected, by way of a microchannel 344c, also referred to as a channel, as well, shown in FIG. 6B. As shown in FIG. 6A, by exciting two pairs of SAWs 380, 381 and 382, 383 propagating asymmetrically across flanks of the droplets 344a, 344b, both of the droplets 344a, 344b can spin simultaneously. The spinning can, but does not have, to be synced between the two droplets 344a, 344b (i.e., the droplets 344a, 344b can have different rotation speeds and can start and stop at different times), as a variety of factors can be controlled to impact a rate of rotation of the droplets 344a, 344b, including but not limited to an amplitude, frequency, angle, and timing of the delivery of the SAWs 380, 381 and 382, 383, among other factors provided for herein or otherwise known to those skilled in the art.

Two acoustic beams can be generated with a single IDT, e.g., IDT 322 or 332, by utilizing a frequency shift keying to switch between two different excitation frequencies and excitation locations along a width of the IDT. More particularly, the centrifuge 310 provides a dual droplet functionality that can be achieved using binary frequency shift keying, which involves sequentially shifting between two frequencies for each IDT. With a high shifting frequency, the two droplets 344a, 344b can be rotated simultaneously. FIG. 6B, which provides a composite image illustrating a particle trajectory or path 349 through a center or microchannel 344c, illustrates that the two droplets 344a, 344b can be connected by the microchannel 344c. The microchannel 344c can serve as a passage for particle transport. More specifically, the passage illustrated in FIG. 6B provides the trajectory 349 along which particles disposed in the droplet 344a traversed to enter the droplet 344b, the trajectory 349 being an approximation of the path traversed, appreciating each particle may have a different path for transport than the other particles. In the non-limiting, illustrated embodiment, the specific frequencies of the waves 383, 380, 381, and 382 can be about 15.3 MHz (f₄), about 15.7 MHz (f₃), about 20.3 MHz (f₂), and about 21.7 MHz (f₁), respectively, with a shifting frequency of about 100 kHz, and f₁>f₂>f₃>f₄.

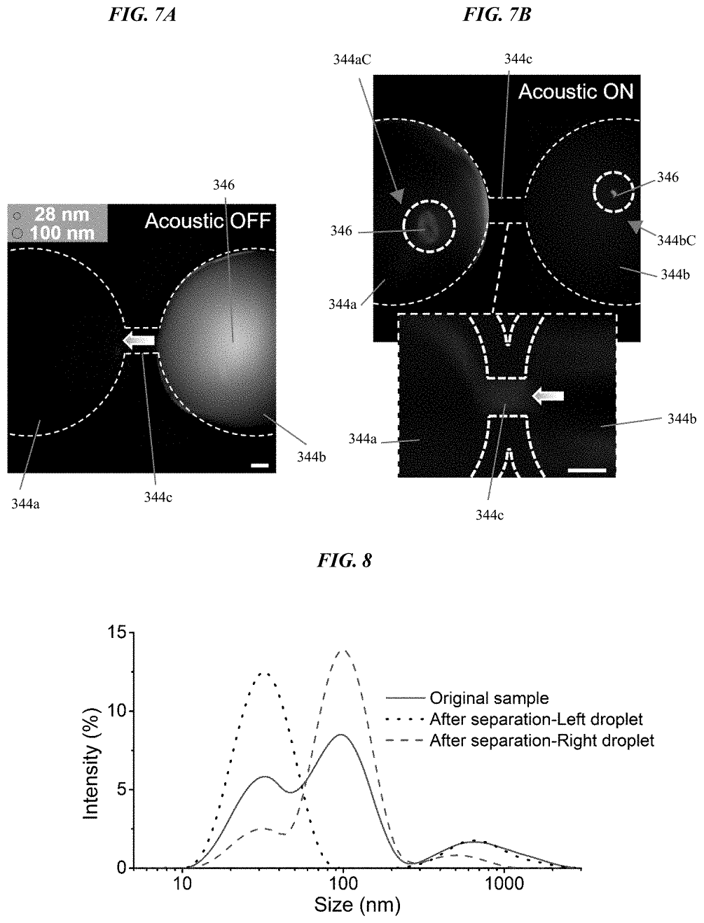

FIGS. 7A and 7B illustrate fluorescent images of the droplets 344a, 344b before and after an acoustic signal is turned on, respectively, showing the nanoparticle separation and transport from one droplet to another. For discussion purposes, all particles referred to in FIGS. 7A and 7B are referred to as particles 346, although it will be understood that with respect to FIGS. 7A and 7B the particles 346 include the particles 347; it is just difficult to differentiate the two particle sizes in the fluorescent images. Further, an inset cc of FIG. 7B provides a further fluorescent image, this time of the middle channel 344c, which helps illustrate the particle transport process. A particle size distribution comparison between the pre and post-separation samples is shown in FIG. 8. The original sample, which was placed into the right droplet 344b, has two peaks at approximately 28 nm and approximately 100 nm. After separation, the majority of the 28 nm particles have been separated and have been transported to the left droplet 344a, which has only one peak at approximately 28 nm. The provided scale bar is 200 μm.

More particularly, for this demonstration, the two droplets 344a, 344b with different volumes can be used (e.g., 4.0 μL and 5.0 μL), which correspond to average rotational speeds of approximately 6,867±160 rpm and approximately 5,674±98 rpm, respectively. Different sized particles 346 (e.g., the two different particles sizes 346, 347 illustrated in FIG. 6A) were initially seeded into the right droplet 344b, while initially the left droplet 344a did not have any particles. After the acoustic waves were turned on (e.g., the waves 380, 381, 382, 383, though not shown in FIGS. 7A and 7B but represented by FIG. 7B being indicated as “Acoustic ON”), particles 346 were separated within the right droplet 344b with the larger particles 346 (equivalent of particles 346 in FIG. 6A) concentrating to a center 344bC and the smaller particles 346 (equivalent of particles 347 in FIG. 6A) remaining dispersed within the right droplet 344b. However, because the spinning velocity of the two droplets 344a, 344b is different, there is a deformation speed difference between the two domains. Hence, there is extra spinning induced radial drag force from the high spin velocity region towards the low velocity region, in addition to convective flow, generated within the fluidic channel 344c between the droplets 344a, 344b. This mechanism allows for the smaller particles 346 (equivalent of particles 347 in FIG. 6A), which are dispersed within the right droplet 344b, to be transferred into the left droplet 344a. Additionally, the acoustic radiation force near the entrance of the right droplet 344b also forces particles 346 into the microchannel 344c towards the left droplet 344a. Experimentally, it was observed that the acoustic beam 381 and 383 (f₂ and f₄) accelerate the particles 346 and push the particles 346 into the microchannel 344c. At the same time, the acoustic beam 380 and 382 (f₁ and f₃), which has a higher frequency and a propagation path closer to a center 344aC of the left droplet 344a, further accelerates the particles 346 exiting from the channel 344c. Interestingly, after the smaller particles 346 (equivalent of particles 347 in FIG. 6A) enter the left droplet 344a, they are, to a certain degree, further concentrated into the middle 344aC of the droplet 344a, as shown in FIG. 7B. This is in contrast to the phenomenon observed in the right (single) droplet 344b, where the 28 nm diameter particles 346 (equivalent of particles 347 in FIG. 6A) remained dispersed as the 100 nm diameter particles 346 (equivalent of particles 346 in FIG. 6A) are concentrated, as shown in FIG. 5. This may reflect the fact that the concentrated larger particles reduce the size of the streaming vortex, which will prevent the concentration of smaller particles.

After separating the two different sized particle distributions using this dual droplet system 310, the separation and transport performance can be characterized by measuring the particle size distribution in both droplets 344a, 344b using a Zetasizer. As shown in FIG. 8, the majority of the remaining particles within the left droplet had diameters beneath 100 nm. This indicates that after the separation, the left droplet contains mostly 28 nm particles, while the majority of the particles in the right droplet were 100 nm particles. It is worth noting that, based on experimental observation and characterization, to achieve high separation yield and purity, the two types of particles would need to have a diameter ratio higher than about 1.5, as the force difference would be large enough to achieve separation instead of simultaneous concentration in one droplet.

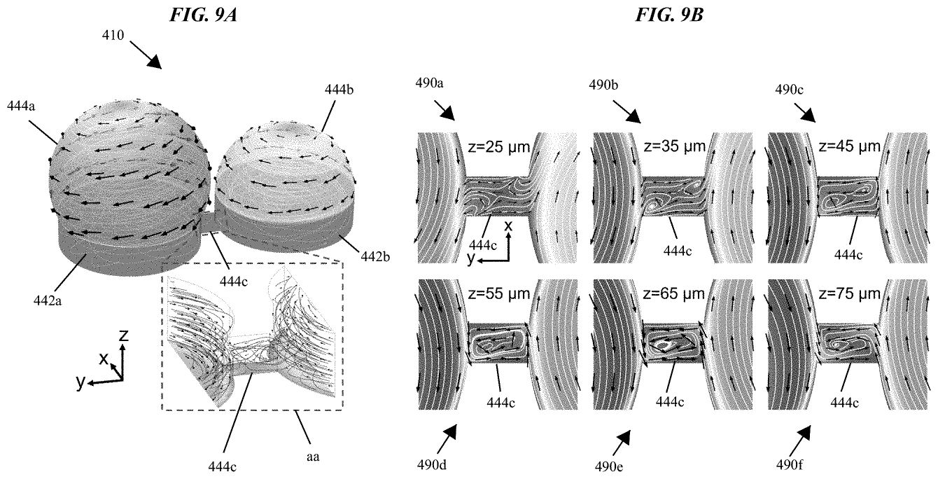

FIGS. 9A-9B illustrate a 3D flow field in a dual-droplet acoustofluidic centrifuge system or device 410, similar to the dual-droplet acoustofluidic centrifuge system 310 described above. More particularly, FIG. 9A provides for a simulated 3D flow field in left and right droplets 444a, 444b, which can also referred to as chambers in the context of at least this simulation, of the dual-droplet system 410. The size and shape of the droplets 444a, 444b can be defined by the containment boundaries 440, as shown rings 442a, 442b, and propagated waves (not shown) can impact the speed at which the droplets 444a, 444b rotate, as explained above. In the illustrated embodiment, the rotation speeds in the droplets 444a, 444b are different, as evidenced by the droplets 444a, 444b having different sized radii, which can assist in causing particles to move from the droplet 444b, through a channel or microchannel 444c (shown more clearly in inset aa of FIG. 9A) formed between the two droplets 444b, 444a, and to the droplet 444a, again as explained above. As shown, both droplets 444a, 444b rotate in the clockwise direction, although other directions are possible. FIG. 9B illustrates the flow fields at different z-planes in the microchannel 444c. More specifically, flow field images 490a, 490b, 490c, 490d, 490e, and 490f represent z being 25 μm, 35 μm, 45 μm, 55 μm, 65 μm, and 75 μm, respectively.

The dual-droplet-based acoustofluidic centrifuge system can also be used to perform extracellular vesicle subpopulation separation. One non-limiting example of such extracellular vesicles includes exosomes, which are nanoscale extracellular vesicles (approximately in the range of about 30 nm to about 150 nm) that carry molecular cargo from their cell of origin. They have emerged as a potentially powerful vector for biomedical research, biomarker discovery, disease diagnostics, and health monitoring. It has been reported that exosomes have three distinct subpopulations (i.e., large exosomes, approximately in the range of about 90 nm to about 150 nm, small exosomes, approximately in the range of about 60 nm to about 80 nm, and exomeres, approximately 35 nm), which exhibit different physical and biological properties. Among these three subpopulations, exomeres, a non-membranous nanoparticle, have the smallest size and distinctive cargos compared to the other two subpopulations. While the recent discovery of exosome subpopulations has excited researchers due to their potential to revolutionize the field of non-invasive diagnostics, exosome subpopulations have yet to be utilized in clinical assays; this is largely due to the difficulties associated with separation of the nano-sized exosome subpopulations.

Because the dual-droplet acoustofluidic centrifuge system, and related methods of using the same, can concentrate and separate nanoparticles with a fine size difference, one non-limiting use for the disclosed systems and methods is to perform exosome subpopulation separation. One example of exosome subpopulation separating is illustrated in FIGS. 10A-10C, described with respect to a system or device 510, which is similar to systems 310 and 410. The system 510 includes a containment boundary 540 that includes two rings 542a, 542b disposed on a substrate 512, with droplets 554a, 554b disposed in the rings 542a, 542b an in fluid communication via a channel or microchannel 554c that forms between the droplets 554a, 554b. As shown in FIG. 10A, an exosome sample, illustrated as larger particles 546 and smaller particles 547, can be placed into the droplet 554b, and, at least to begin, pure PBS can be placed in the droplet 554a. Acoustic waves (not shown) can be provided in accordance with the present disclosures, causing acoustic wave excitation that results in the droplets 554a, 554b rotating. In the illustrated embodiment, both droplets 554a, 554b rotate in a counterclockwise direction, although other rotation profiles are possible. As a result, as shown in FIG. 10B, larger particles 546 can be concentrated to an approximate middle or center 554bC of the droplet 554b and the smaller particles 547 can gradually be transferred to the other droplet 554a, as illustrated by arrow 548. After the separation and transport process, as shown in FIG. 10C, particles 546, 547 with different sizes can be immersed in different droplets 554a, 554b, the smaller ones 547 fully in the droplet 554a and the larger ones 546 fully in the droplet 554b. Pipettes 560, 562, as shown two, can be used to extract the sample 547, 546 from the droplets 554a, 554b, respectively.

The samples 546, 547 can be measured using a nanoparticle tracking analysis (NTA) system. In the original sample, there can be multiple peaks within the size range of the exosome subpopulations beneath the 150 nm size range. After the separation and transport process, however, there can be two major size distribution peaks that remain in the droplet 554b, both the larger particles 546 and the smaller particles 547. Meanwhile, in the droplet 554a, the majority of the particles are the smaller particles 547, which can measure below 50 nm. The structures within the three different samples (original, right droplet 554b after separation, left droplet 554a after separation) were TEM imaged, which supported the results of the NTA measurements. The images showed a larger percentage of exomeres in the left droplet 554a, again supporting the NTA measurements. These results demonstrate that this dual-droplet acoustofluidic centrifuge system 510, and other such systems disclosed herein (e.g., systems 310, 410) or otherwise derivable from the present disclosure, can be used to rapidly fractionalize exosome samples into different subpopulations. It is worth noting that the peaks of the particle size distribution from NTA measurement can have small shifts before and after separation. This phenomenon may be due, at least in part, to the blocking effect of the intense light scattering of large particles as the size of the particle distributions are different in pre- and post-separation sample. This insufficient polydisperse sample resolution, although difficult to avoid, may be reduced by minimizing the large nanoparticle effect from particles with size ranges out of a realm of interest.

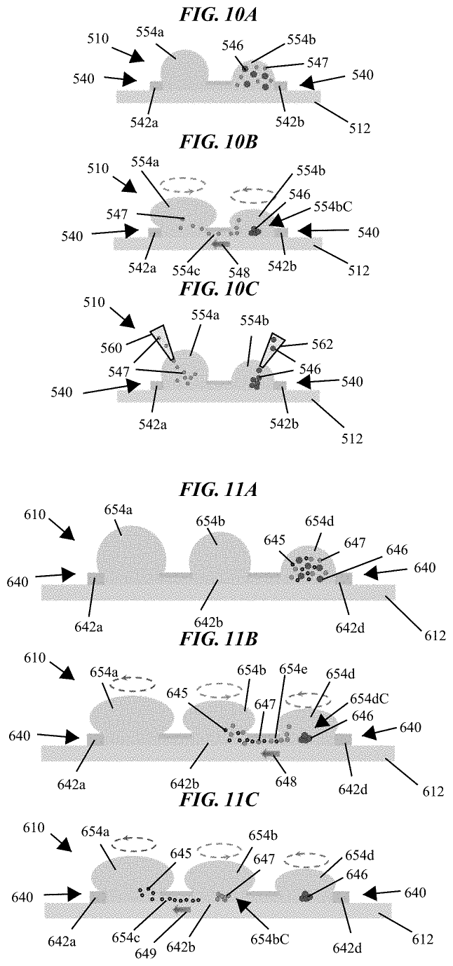

FIGS. 11A-11C provide for a similar process as FIGS. 10A-10C, but for an instance in which a multi-droplet acoustofluidic centrifuge system 610 provides for three droplets 654a, 654b, 654d, and thus a containment boundary 640 that includes three rings 642a, 642b, 642d. The rings 642a, 642b, 642d can be disposed on a substrate 612, and channels or microchannels 654c, 654e can be form between the droplets 642a, 642b and 642b, 642d, respectively, as illustrated. As shown in FIG. 11A, a sample (e.g., an exosome sample, though the present disclosure provides for other samples that can be used, and a person skilled in the art will appreciate other samples that can be used), illustrated as larger particles 646, medium-sized particles 647, and smaller particles 645, can be disposed in the droplet 654d. After excitation is initiated, for example by using acoustic waves (not shown), the larger 646 particles can be concentrated to an approximate middle or center 654dC of the right droplet 654d and the medium-sized particles 647 and smaller particles 645 can gradually be transferred to the middle droplet 654b, as illustrated by arrow 648 shown in FIG. 11B. In the illustrated embodiment, all three droplets 654a, 654b, 654d rotate in a counterclockwise direction, although other rotation profiles are possible. Additional transport can occur, further separating the medium-sized particles 647 and the smaller particles 645, the former of which can be concentrated to an approximate middle or center 654bC of the middle droplet 654b, and the small particles 645 can gradually be transferred to the left droplet 654a, as illustrated by arrow 649 shown in FIG. 11C. Although not illustrated, samples can be removed, such as by using pipettes and/or other tools and techniques known to those skilled in the art, similar to what is illustrated in FIG. 10C.

### SUMMARY

The acoustofluidic centrifuges, and related methods, provided for herein can use SAWs to push a droplet out of its equilibrium shape and forces it to spin on its center vertical axis when the fluid boundary is partially confined. This rotational velocity can be independent of the acoustic parameters, but can be closely related to the radius of the droplet. This can allow the spin rate to be tuned, for example by either changing the volume of the droplet or the size of the PDMS containment ring. Meanwhile, this spin motion can also be extended to different organic liquids that are often used for cell/nanoparticle handling (e.g., PBS, cell culture medium, or Bovine serum albumin). In a spinning droplet, Stokes drift rotates and forms a closed path creating a helical trajectory that particles inside the droplet will follow towards the droplet center. With the acoustofluidic centrifuge system, high-frequency acoustic waves will propagate into the fluid and generate acoustic streaming and acoustic radiation forces which both act on the particles and push the particles towards the center of the droplet, as opposed to along specific orbits. The spinning of the droplet not only enables the helical trajectory, but also increases the speed of particle concentration by a factor approximately in the range of about 10² time to about 10³ times by significantly enhancing the streaming velocity along with the secondary-flow induced radial drag force for nanoparticles. All of these phenomena can form an interesting and functional system that can bridge the gap between acoustofluidics and nanoscale bioparticle manipulation.

Based on experiments and numerical simulations, it has been shown that particles with sizes ranging from several nanometers (i.e., DNA molecules) to micrometers can be rapidly concentrated in acoustofluidic centrifuge systems provided for herein. Furthermore, a practical method to separate nanoparticles with different size distributions was demonstrated using a dual-droplet acoustofluidic centrifuge system. This dual-droplet system can be further expanded to a multi-stage nanoparticle separation or enrichment system in which multiple (greater than 2) groups of nanoparticles can be separated into different outlets in one device. Many bioparticles have sizes that range in the nanoscale or submicron (e.g., DNA, exosomes, bacteria, proteins, or viruses), and the enrichment and separation of these bioparticles is of great importance in biology, chemistry, and medicine. However, the current methods for nanoparticle enrichment and separation, such as ultracentrifugation, often require a large sample volume (typically greater than about 10 mL) and a long processing time (typically at least several hours to several days) with relatively low yield (approximately in the range of about 5% to about 50%) and purity (approximately in the range of about 23% to about 70%). By combining acoustic waves and fluid motion, the disclosed acoustofluidic centrifuges possess advantages in both of these regards. The devices can flexibly handle smaller sample volumes ranging from nanoliters to microliters, and processing times (approximately one minute or less) are much shorter than currently available nanoparticle concentration/separation mechanisms. Further, the systems can sustain a relatively high yield and purity (approximately greater than 80%). Furthermore, it is an open microfluidic device with a simple fabrication process. This allows for easy accessibility to the droplet via a pipette, eliminating the need for external pumps, valves, or other flow control devices.

The effects of different factors involved in the operation of the disclosed acoustofluidic centrifuges have been explored to better understand and improve the device performance. For example, if the streaming within the droplet is too strong, it may induce the re-dispersion of concentrated particles, especially in smaller droplets, which have a high rotational velocity. On the other hand, if the streaming is too weak, it may cause insufficient enrichment. Thus, device-operating parameters should generally be optimized for particular applications. In the disclosed acoustofluidic centrifuges, many factors should be considered for optimizing nanoparticle motion within the rotational vortex (e.g., Brownian motion, the extra shear rate, and the secondary-flow drag force in the spinning droplet). In this regard, numerical simulations were performed to investigate the interaction between these factors. This simulation model is presented as an efficient tool to estimate the proper streaming velocity and radial force that is needed to concentrate/separate nanoparticles with different size distributions, and the trajectories of different sized particles could be simulated and traced as well. Moreover, the numerical simulation can be used to isolate the effects of droplet spinning motion from acoustic streaming, which is difficult to observe during experimentation because the particle motion within the droplet is an intertwined effect combining these two motions.

In summary, the present disclosure provides for an acoustofluidic centrifuge platform (both systems and methods) that can efficiently and rapidly enrich or separate nanoscale bioparticles. This platform can significantly simplify and speed up sample processing, detection, and reagent reactions in various applications such as point-of-care diagnostics, bioassays, and liquid biopsies.

Additional details about some of the components disclosed herein are provided for in the following listing of materials and methods. The identification of particular materials, parameters, etc. are by no means limiting, but provide only a non-limiting embodiment(s) of components of the acoustofluidic centrifuge system provided for herein.

Device design, fabrication and operation. The slanted IDTs (5-nm Cr and 50-nm Au) can be fabricated on a 128° Y-cut lithium niobite (LiNbO₃) wafer (Precision Micro-Optics, USA) using standard photo lithography followed by electron beam evaporation and a lift-off process. Silver epoxy (MG Chemicals, USA) can be used to connect wires to the IDT electrodes. The microchannels can be fabricated by standard soft-lithography and a mold-replica procedure. The containment boundaries, e.g., PDMS rings, can be cut to the desired size from a 0.55 mm-thick PDMS film using punches (Robbins Instruments, USA). The PDMS parts and LiNbO₃ substrate can be bonded together after approximately three minutes of treatment in an oxygen plasma cleaner (Harrick Plasma, USA). Three slanted IDT configurations used in the setup corresponded to different droplet volume ranges (approximately 10 μL, approximately 1 μL, and approximately 100 nL). The first has an electrode finger width and spacing gap which decreases linearly from about 140 μm to about 70 μm, corresponding to SAW frequencies from about 7 MHz to about 14 MHz. The second has finger widths from about 75 μm to about 35 μm, corresponding to about 13 MHz to about 28 MHz. The third has widths from about 32.5 μm to about 17.5 μm, corresponding to about 30 MHz to about 56 MHz. Two function generators (DG 3012C, Teletronics Technology Corporation, USA) and two amplifiers (25A250A, Amplifier Research, USA) can be used to activate a pair of slanted IDTs and to generate SAWs. For the acoustofluidic centrifuge system with dual spinning droplets, the microchannel can be designed with a width of about 200 μm and a height of about 100 μm.

Droplet generation and sample preparation. The micro droplets can be generated using a pipette and the nano droplets can be generated using 1 μL and 0.5 micro-volume liquid syringes (SEG, AU). 10 μm, 5 μm, 1 μm, 970 nm, 530 nm, 100 nm, 51 nm, and 28 nm diameter polystyrene particles (Sigma-Aldrich, USA, Bangs Laboratories, USA) with different fluorescence tags can be utilized in the experiments. Deoxyribonucleic acid chains from herring sperm in solution (Sigma-Aldrich, USA) can be tested after staining with SYTOX orange dye (Thermo Fisher, USA).

Small EV preparation procedure. The exosome sample can be isolated from human plasma (Zen-Bio, USA) with a concentration of about 10⁶ /mL using differential ultracentrifugation. One non-limiting embodiment of the general procedure can be:
1. Thaw plasma in an approximately 37° C. water bath until all, or substantially all, the crystals of ice in the tubes have disappeared. After thawing is complete, mix the samples by gently vortexing for about 10 seconds.
2. Centrifuge about 45 mL of plasma at about 3,000 g for about 10 minutes at approximately 8° C.
3. Dilute cleared plasma with PBS and spin at about 10,000 g for about 30 minutes at approximately 8° C. Re-suspend and collect 10K pellet in about 1.0 ml PBS per tube.
4. Wash re-suspended pellets using an approximately 30 minute spin at about 10,000 g at approximately 8° C.
5. Spin supernatant at about 103,745 g for about 4.5 hours at approximately 8° C. Re-suspend and collect 100K pellet in about 0.5 ml PBS per tube.
6. Combine re-suspended plasma 100K pellets (about 2 ml total), add about 4 ml PBS, and place on top of a 3-part OptiPrep cushion (about 2 ml 50%/about 2 ml 30%/about 2 ml 10%). Centrifuge at about 178,000 g, about 2 hours at approximately 4° C. Collect about 1 ml of the 30%/10% interface (p=about 1.06 g/ml to about 1.16 g/ml).
7. Dilute OP about 30%/about 10% interface to about 12 ml with PBS and spin at about 120,000 g for about 2 hours at approximately 8° C.
8. Re-suspend pellets in about 200 μl PBS and analyze using NTA for confirmation.

Image acquisition and analysis. The microscope images and videos can be acquired using an inverted microscope (TE2000-U, Nikon, Japan) equipped with a fast camera (Photron, Japan). The droplet spinning motion can be captured with a frame rate of about 3,000 fps and analyzed using ImageJ (NIH, MD, USA) and/or MATLAB R2016b (MathWorks, USA). The side view of the droplet spinning can be captured using the Slow Mo mode of a cell phone with a frame rate of about 240 fps. The post-processed exosome sample can be collected and visualized using transmission electron microscopy (TEM, FEI Tecnai G² Twin, FEI Company, USA) and a negative staining method. The nanoparticle size distribution and concentration pre- and post-processing can be analyzed using the Malvern Zetasizer (Malvern Instruments, UK) and nanoparticle tracking analysis (NTA) with a NanoSight LM10 apparatus (Amesbury, UK).

The systems and methods described herein can be implemented in hardware, software, firmware, or combinations of hardware, software and/or firmware. In some examples, the systems and methods described in this specification may be implemented using a non-transitory computer readable medium storing computer executable instructions that when executed by one or more processors of a computer cause the computer to perform operations. Computer readable media suitable for implementing the systems and methods described in this specification include non-transitory computer-readable media, such as disk memory devices, chip memory devices, programmable logic devices, random access memory (RAM), read only memory (ROM), optical read/write memory, cache memory, magnetic read/write memory, flash memory, and application-specific integrated circuits. In addition, a computer readable medium that implements a system or method described in this specification may be located on a single device or computing platform or may be distributed across multiple devices or computing platforms.

One skilled in the art will readily appreciate that the present disclosure is well adapted to carry out the objects and obtain the ends and advantages mentioned, as well as those inherent therein. Further, one skilled in the art will appreciate further features and advantages of the disclosure based on the above-described embodiments. Accordingly, the disclosure is not to be limited by what has been particularly shown and described, except as indicated by the appended claims.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

What is claimed is:

1. An acoustofluidic centifuge system, comprising:
at least one sound wave generator; and
at least one containment boundary configured to encapsulate at least a portion of a fluid droplet,
wherein the sound wave generator is configured to generate acoustic waves that propagate towards the at least one containment boundary to cause the at least a portion of a fluid droplet encapsulated therein to spin along a central axis.

2. The system of claim 1, wherein the at least one sound wave generator comprises at least one interdigitated transducer.

3. The system of claim 2, wherein the at least one interdigitated transducer is slanted.

4. The system of claim 2, wherein the at least one interdigitated transducer comprises a pair of opposed interdigitated transducers, each transducer of the pair being disposed on opposite sides of the at least one containment boundary.

5. The system of claim 4, wherein the pair of opposed interdigitated transducers are slanted.

6. The system of claim 1, wherein the at least one containment boundary comprises a circular ring.

7. (canceled)

8. The system of claim 1, wherein the at least one containment boundary comprises two containment boundaries, the two containment boundaries being in communication with each other by way of a channel disposed therebetween.

9. (canceled)

10. A method of separating nanoparticles, comprising:
generating at least one sound wave such that the sound wave propagates to at least one containment boundary having at least a portion of one fluid droplet disposed therein, the at least one fluid droplet having a plurality of nanoparticles disposed therein;
wherein the at least one sound wave causes the at least a portion of one fluid droplet to rotate, and
wherein rotation of the at least a portion of one fluid droplet causes at least a first nanoparticle of the plurality of nanoparticles to travel along a first trajectory of a plurality of trajectories and at least a second nanoparticle of the plurality of nanoparticles to travel along a second trajectory of the plurality of trajectories, the first and second trajectories being different traj ectories.

11. The method of claim 10, wherein generating at least one sound wave further comprises providing current to at least one acoustic transducer, which propagates at least one acoustic wave to the at least one containment boundary.

12. The method of claim 10, wherein generating at least one sound wave further comprises providing current to at least one interdigitated transducer, which propagates at least one acoustic wave to the at least one containment boundary.

13. The method of claim 10, wherein the at least one sound wave deforms a liquid-air interface of the at least a portion of one fluid droplet, resulting in the at least a portion of one fluid droplet to rotate.

14. The method of claim 10, wherein at least one trajectory of the plurality of trajectories along which at least one nanoparticle of the plurality of nanoparticles travels comprises a helical path.

15. The method of claim 13, wherein at least one of the at least one nanoparticle that travels along the first trajectory and the at least one nanoparticle that travels along the second trajectory travels along a vortex-shaped streamline and is influenced by an angular momentum of the rotation of the at least a portion of one fluid droplet.

16. The method of claim 10, wherein the rotation of the at least a portion of one fluid droplet has a dual-axis rotational trajectory.

17. The method of claim 10, further comprising controlling an acoustic streaming speed of the at least a portion of one fluid droplet.

18. The method of claim 16, wherein controlling an acoustic streaming speed of the at least a portion of one fluid droplet further comprises controlling at least one of an acoustic wave amplitude, a frequency of an acoustic wave, an angle at which an acoustic wave is delivered, and a location of the acoustic wave.

19. (canceled)

20. The method of claim 10, wherein travel along the first trajectory or the second trajectory is based on a size of the nanoparticles of the plurality of nanoparticles.

21. The method of claim 10,
wherein the at least one containment boundary comprises a plurality of containment boundaries in fluid communication with each other, the plurality of containment boundaries comprising a first containment boundary and a second containment boundary,
wherein the at least one sound wave causing the at least a portion of one fluid droplet to rotate further comprises causing a first at least a portion of one fluid droplet to rotate in the first containment boundary and causing a second at least a portion of one fluid droplet to rotate in the second containment boundary, and
wherein causing at least a first nanoparticle of the plurality of nanoparticles to travel along a first trajectory and causing at least a second nanoparticle of the plurality of nanoparticles to travel along a second trajectory comprises passing at least one of the first nanoparticles or the second nanoparticles through a channel disposed between the first containment boundary and the second containment boundary.

22. (canceled)

23. A method of isolating different extracellular vesicle subpopulations, comprising:
mixing DNA strands disposed in at least a portion of a fluid droplet with a fluorescent marker;
intercalating the at least a portion of a fluid droplet such that the DNA strands are able to express a fluorescent signal; and
activating an acoustic signal to activate the fluorescent marker in a manner such that concentrated DNA strands express an amplified fluorescent signal.

24. The method of claim 23, wherein activating an acoustic signal to activate the fluorescent marker in a manner such that concentrated DNA strands express an amplified fluorescent signal further comprises:
operating at least one sound wave generator to produce the acoustic signal,
wherein the at least a portion of a fluid droplet is encapsulated by at least one containment boundary, and
wherein the acoustic signal is effective to cause the at least a portion of a fluid droplet to spin along a central axis of at least one of the at least a portion of a fluid droplet or the containment boundary.

25. (canceled)

26. The method of claim 23, wherein activating an acoustic signal further comprises generating at least one sound wave such that the sound wave propagates to at least one containment boundary having the at least a portion of one fluid droplet disposed therein.

27. (canceled)
